(19) United States
(12) Patent Application Publication
(10) Pub. No.: US 2024/0317750 A1
(43) Pub. Date: Sep. 26, 2024

(54) An Efficient Process for the Synthesis of Methylliberine and Polymorphs Thereof

(71) Applicant: Fertis India Pvt. Ltd., Hyderabad (IN)

(72) Inventors: Rahul Raju Kanumuru, Hyderabad (IN); George Kochumalayil Shaji, Hyderabad (IN); Murali Mohanarao Vana, Hyderabad (IN); Ravikumar Suraneni, Hyderabad (IN); Sil Anindya, Hyderabad (IN)

(21) Appl. No.: 18/573,801

(22) PCT Filed: Mar. 22, 2022
(86) PCT No.: PCT/IN2022/050279

§ 371 Date: Dec. 22, 2023

(30) Foreign Application Priority Data
Jun. 24, 2021 (IN) 202141028486

(51) Int. Cl. C07D 473/06; C07C 273/02; C07D 239/52

(52) U.S. Cl. CPC C07D 473/06; C07C 273/02; C07D 239/52

(57) ABSTRACT

The present invention discloses a synthetic procedure for the preparation of Methylliberine and its polymorphs thereof in a simple and efficient manner.

### FIELD OF THE INVENTION

The present invention relates to a synthetic process for the preparation of Methylliberine and its polymorphs thereof in a simple and efficient manner.

### BACKGROUND OF THE INVENTION

Methylliberine, a methoxyuric acid, is a caffeine metabolite present at low levels in various Coffee plants. Chemically, Methylliberine is 2-Methoxy-1,7,9-trimethyl-7,9-dihydro-1H-purine-6,8-dione having the following structure;

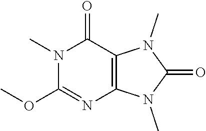 [SMILES: COc1nc2c(c(=O)n1C)n(C)c(=O)n2C]

In recent years, studies have found that due to structural similarities to the methylxanthine, caffeine and methylliberine possess similar physiological properties but without undesirable stimulant effects. The available toxicological information on the molecule shows that methylliberine does not cause any genotoxicity and possible health hazards and there is continuous interest in methylliberine as an ingredient in functional foods and dietary supplements.

This purine alkaloid is low in content in coffee plants and the extraction process is cumbersome. From the reports in the literature, it is observed that biotransformation of natural raw materials such as caffeine into methylliberine compounds is expensive, the efficiency is low, and it is difficult to commercialize due to poor atom economy. The present invention provides relatively high yield of methylliberine and the process is relatively inexpensive.

Regarding the chemical synthesis of Methylliberine (5), there are basically few reports in the art. Only in Phytochemistry 1975, 14, 747-750, the method of 0(2)-methyluric acid methylation is reported which has no industrial value.

Available synthetic method by Zhang, Jian, Liao and Qilin in the patent Faming Zhuanli Shenquing; 108912121 states; 6-amino-2-methoxy pyrimidin-4(3H)-one upon methylation, nitrosation, reduction, cyclisation and methylation yields the desired methylliberine. This method is 7 step synthetic approach and involves usage of noxious ammonium sulfide and toxic metals for reduction of nitroso compound. The major disadvantage of the method is that methylation is conducted in two stages.

The present inventors therefore felt that the unfilled gap in the production of Methylliberine on industrial scale can be met by hazard free chemical synthesis of Methylliberine (5) in a cost-effective manner with high atom economy.

### SUMMARY OF THE INVENTION

In lieu of the above objective, the present invention provides a process for the synthesis of Methylliberine via the intermediate (4) in an efficient manner comprising;
(a) Reacting 6-amino-2-methoxy-5-nitrosopyrimidin-4-(3H)-one (3) dissolved in formic acid with glyoxilic acid to form the intermediate (3a) which is transformed to intermediate (3b) with subsequent hydroxylation and simultaneous decarboxylation of intermediate (3b) to yield 2-methoxy-1H-purine-6,8-(7H,9H)-dione (4); and
(b) Methylating intermediate (4) in presence of base to obtain methylliberine (5).

In an aspect, the intermediate (3) is obtained from urea by the process comprising;
(a) converting urea (1) to O-methylisoureahemisulfate (1a), followed by in-situ cyclization with ethyl cyanoacetate to obtain 6-amino-2-methoxy-pyrimidin-4(3H)-one (2); and
(b) Nitrosating 6-amino-2-methoxy-pyrimidin-4(3H)-one (2) to obtain 6-amino-2-methoxy-5-nitrosopyrimidin-4(3H)-one (3).

In yet another aspect, the process for preparation of methylliberine (5) of the present invention comprises the steps of;
(a) Converting urea (1) to O-methylisoureahemisulfate (1a), followed by insitu cyclization with ethyl cyanoacetate to obtain 6-amino-2-methoxy-pyrimidin-4(3H)-one (2);
(b) Nitrosating 6-amino-2-methoxy-pyrimidin-4(3H)-one (2) to obtain 6-amino-2-methoxy-5-nitrosopyrimidin-4(3H)-one (3);
(c) Reacting 6-amino-2-methoxy-5-nitrosopyrimidin-4(3H)-one (3) dissolved in formic acid with glyoxilic acid to form the intermediate (3a) which is transformed to intermediate (3b) with subsequent hydroxylation and simultaneous decarboxylation of intermediate (3b) to yield 2-methoxy-1H-purine-6,8(7H,9H)-dione (4); and
(d) Methylating intermediate (4) in presence of a base to obtain methylliberine (5).

In an aspect, the present invention discloses the compound 6-amino-2-methoxy-4(3H)-one of formula (2),

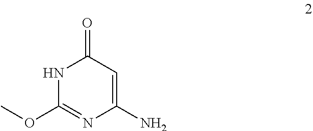

In another aspect, the present invention discloses the novel intermediate 6-amino-2methoxy-5-nitrosopyrimidin-4(3H)-one of formula (3)

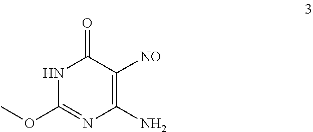

In another embodiment, the present invention discloses the novel intermediate of the formula (4),

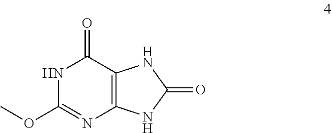

In an aspect, the present invention provides a process for the preparation of polymorphs of methylliberine (5) which comprises crystallization of the prepared methylliberine (5) from a suitable solvent selected from lower alcohols such as ethanol, methanol, isopropanol, propanol, n-butanol, tert-butanol; halogenated hydrocarbons such as methylene dichloride, ethylene dichloride; ketones such as acetone, methyl ethyl ketone; ethers such as diethylether, THF; hydrocarbons such as hexane, heptanes, toluene, xylene; and such like alone or mixtures thereof.

### DETAILED DESCRIPTION OF THE INVENTION

The present invention will now be explained in detail with reference to its various preferred as well as optional embodiment, which, however should not be construed to limit the scope of the invention.

In an embodiment, the present invention relates to a process for the preparation of Methylliberine (5) via the intermediate (4) in an efficient manner which comprising;
(a) Reacting 6-amino-2-methoxy-5-nitrosopyrimidin-4(3H)-one (3) dissolved in formic acid with glyoxilic acid to form the intermediate (3a) which is transformed to intermediate (3b) with subsequent hydroxylation and simultaneous decarboxylation of intermediate (3b) to yield 2-methoxy-1H-purine-6,8-(7H,9H)-dione (4); and
(b) Methylating intermediate (4) in presence of base to obtain methylliberine (5).

In an embodiment, the intermediate (3) is obtained from urea by the process comprising;
(a) reacting urea (1) with dimethyl sulfate and sulfuric acid to obtain O-methylisourea hemisulfate (1a), followed by in-situ cyclization with ethyl cyanoacetate to obtain 6-amino-2-methoxy-pyrimidin-4(3H)-one (2); and
(b) Nitrosating 6-amino-2-methoxy-pyrimidin-4(3H)-one (2) to obtain 6-amino-2-methoxy-5-nitrosopyrimidin-4(3H)-one (3).

In an embodiment, the process for preparing methylliberine (5) in an efficient manner comprises the steps of;
(a) Reacting urea (1) with dimethyl sulfate and sulfuric acid to obtain O-methylisoureahemisulfate (1a) followed by in-situ cyclization with ethyl cyanoacetate to obtain 6-Amino-2-methoxy-pyrimidin-4(3H)-one (2);
(b) Nitrosating 6-amino-2-methoxy-pyrimidin-4(3H)-one 2 to obtain 6-amino-2-methoxy-5-nitrosopyrimidin-4-(3H)-one (3);
(c) Reacting 6-amino-2-methoxy-5-nitrosopyrimidin-4(3H)-one (3) dissolved in formic acid with glyoxilic acid to form the intermediate (3a) which is transformed to intermediate (3b) with subsequent hydroxylation and simultaneous decarboxylation of intermediate (3b) to yield 2-methoxy-1H-purine-6,8(7H,9H)-dione (4); and
(d) Methylating intermediate (4) in presence of base to obtain methylliberine (5).

Schematically the process is as follows:

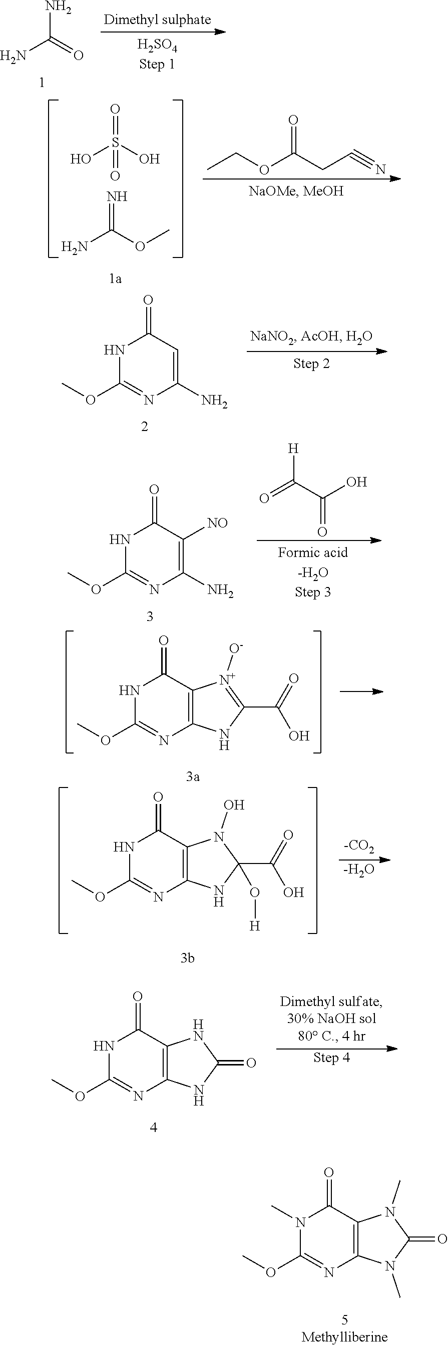

Accordingly, the process step 1 comprises reaction between dimethyl sulfate and urea in presence of 50% sulfuric acid at about 70° C., resulting in the formation of O-Methyl isourea (1a) which upon in-situ cyclization with ethyl cyanoacetate in presence of sodium methoxide gives 6-amino-2-methoxy-pyrimidin-4-(3H)-one (2).

The process step 2 comprises nitrosation of 6-amino-2-methoxy-pyrimidin-4-(3H)-one (2) using nitrosating agent sodium nitrite and 50% acetic acid, to afford 6-amino-2-methoxy-5-nitrosopyrimidin-4(3H)-one (3).

The process step 3 of the present process comprises adding glyoxylic acid to a stirred solution of 6-amino-2-methoxy-5-nitrosopyrimidin-4-(3H)-one (3) in formic acid and heating the mixture to about 50° C. for about an hour. Cooling the mixture to 0° C. and the precipitated solid is filtered and washed to obtain the dione intermediate (4).

The process step 4 of the present process comprises of adding dimethyl sulphate and NaOH solution in Methanol to the dione intermediate (4) to afford Methylliberine (5).

The methylating reagent for step 4 is selected from dimethyl sulfate or dimethyl carbonate or N,N-dimethylformamide-dimethylacetal (DMF-DMA), trimethyl phosphate or any such suitable methylating agent. The base is selected from alkali or alkaline metal hydroxides, carbonates or bicarbonates, alkali or alkaline methoxides such as sodium methoxide, sodium ethoxide, sodium tert-butoxide, potassium tert-butoxide, or diethylamine, triethylamine, diisopropylethylamine or pyridine or 1,8-diazabicyclo [5.4.0] undec-7-ene (DBU) or 1,5-diazabicyclo[4.3.0]non-5-one.

The solvent for the present process is selected from polar or non-polar, protic or aprotic organic solvents such as DMF, DMA, ketones, ethers, esters, lower aliphatic or aromatic hydrocarbons, lower alcohols alone or mixtures thereof.

The present invention provides an efficient process for synthesis of methylliberine (5), avoids toxic chemicals and solvents and results in the formation of intermediate and the end product in good atom economy and high purity.

In another embodiment, the present invention discloses the compound 6-amino-2-methoxy-4(3H)-one of formula (2),

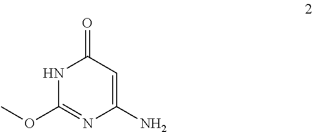

In another embodiment, the present invention discloses the compound 6-amino-2-methoxy-5-nitrosopyrimidin-4(3H)-one of formula (3),

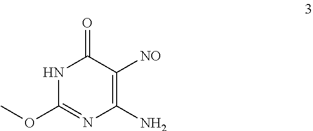

In yet another embodiment, the present invention discloses the compound of the formula (4),

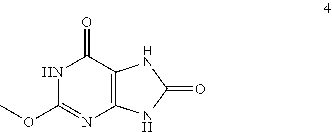

In yet another embodiment the present invention discloses a process for the preparation of polymorphs of methylliberine (5) which comprises crystallization of the as prepared methylliberine (5) from a suitable solvent selected from water, lower alcohols such as ethanol, methanol, isopropanol, propanol, n-butanol, tert-butanol; halogenated hydrocarbons such as methylene dichloride, ethylene dichloride; ketones such as acetone, methyl ethyl ketone; ethers such as diethylether, THF; hydrocarbons such as hexane, heptanes, toluene, xylene; and such like alone or mixtures thereof.

### EXPERIMENTAL

### Example 1: Preparation of 6-Amino-2-methoxy-pyrimidin-4(3H)-one (2)

In a 20 L three-necked flask equipped with a mechanical stirrer, heating bath, reflux condenser and drying tube a mixture of dimethyl sulfate (700 g, 5.55 mol) and 50% aqueous sulfuric acid (7 g, 0.071 mol) were added, followed by lot wise addition of urea (333.3 g, 5.55 mol) at 70° C. Reaction mixture was continued to stir for that same temperature. Reaction mixture was then cooled to 60° C., followed by addition of urea (333.3 g, 5.55 mol) and dropwise addition of dimethyl sulfate (700 g, 5.55 mol) at 70° C. The process was repeated twice, i.e., urea and dimethyl sulfate were added twice in same quantities in the manner described. Reaction mixture was cooled to room temperature, followed by addition of Methanol (12 L), and sodium methoxide (1.65 kg, 30.73 mol). Reaction mixture was stirred at room temperature for 10 min and added ethyl cyanoacetate (1.73 kg, 15.37 mol). The reaction mixture was stirred at 80° C. for 3 h and was cooled to room temperature. Reaction mixture was filtered and volatiles were evaporated to dryness. The residue thus obtained was dissolved in water and acidified with slow addition of acetic acid, until the solution became acidic (pH˜6). The solid precipitated was filtered, washed with water and dried under vacuum at 40-45° C. to afford 6-Amino-2-methoxy-pyrimidin-4(3H)-one (2), as an off-white solid. Yield: 800 g, (34%); mp 202-204° C.; ¹HNMR (400 MHz, DMSO-d₆): 53.78 (3H, s), 4.72 (OH, s), 6.39 (2H, bs), 11.12 (1H, bs). ¹³C NMR (400 MHz, DMSO-d₆): 54.1, 79.3, 158.2, 164.2. MS(m/z): 141.97 (M+H)⁺. IR: vKBr: 3326, 3249, 1651, 1620, 1343 and 1162 cm⁻¹.

### Example 2: Preparation of 6-amino-2-methoxy-5-nitrosopyrimidin-4(3H)-one (3)

To a stirred solution of 6-amino-2-methoxypyrimidin-4(3H)-one (2) (970 g, 6.87 mol), obtained in example 1, in water (9.7 L), was added sodium nitrite (570 g, 8.25 mol), followed by slow addition of acetic acid (1.9 L). The reaction mixture was stirred for 2 h at 50° C. The reaction mixture was cooled to room temperature and the solid thus precipitated was filtered and washed with cold water to afford 6-amino-2-methoxy-5-nitrosopyrimidin-4-(3H)-one (3), as a bright purple solid. Yield: 830 g (71%). ¹HNMR (400 MHz, DMSO-d6): δ 3.84 (3H, s), 11.65 (1H, bs). ¹³C NMR (400 MHz, DMSO-d6): δ 53.8, 144.1, 154.1, 169.2, 173.8. MS (m/z): 171.04 (M−H). IR: vKBr: 3489, 3478, 1681, 1617, 1505, 1303 and 1118 cm⁻¹.

### Example 3: Preparation of 2-methoxy-1H-purine-6,8(7H,9H)-dione (4)

To a stirred solution of 6-amino-2-methoxy-5-nitrosopyrimidin-4(3H)-one (3) (670.0 g, 3.95 mol), obtained in example 2, in water (3.35 L), was added formic acid (640 g, 11.8 mol), followed by dropwise addition of 50% glyoxylic acid (583 g, 7.88 mol) at 50-55° C. The reaction mixture was stirred at 50° C. for 1 h and cooled to room temperature. The precipitate was collected by filtration and washed with cold water to afford 2-methoxy-1H-purine-6,8(7H,9H)-dione (4) as off-white solid. Yield: 520 g (73%). ¹HNMR (400 MHz, DMSO-d6): δ 3.85 (3H, s), 10.63 (1H, s), 11.27 (1H, s), 12.18 (1H, bs). ¹³C NMR (400 MHz, DMSO-d6): δ 54.9, 102.8, 145.5, 151.4, 153.0, 155.2. MS(m/z): 180.95 (M−H). IR: vKBr: 3457, 3471, 2346, 1711, 1665, and 1155 cm⁻¹.

### Example 4: Preparation of 2-Methoxy-1,7,9-trimethyl-7,9-dihydro-1H-purine-6,8-dione (Methylliberine 5)

To a stirred solution of intermediate (4) (510.0 g, 2.80 mol), obtained in example 3, in MeOH (5 L) were added 30% aq.NaOH solution (672.0 g, 16.8 mol) followed by addition of dimethyl sulfate (1.5 L, 16.8 mol). The reaction mixture was stirred at 50° C. for 4 h. After 4 h, the reaction mixture was cooled to room temperature added water and extracted with DCM (3×1000 mL). The combined organic layers were dried over Na₂SO₄ and concentrated under vacuum. The crude was dissolved in MeOH (5 vol) and heated at reflux for 2 h, then cooled at 40° C., solid formed was filtered, washed with hot MeOH (1 vol, 40° C.), to afford Methylliberine (5) as white solid. Yield: 350 g (56%). ¹HNMR spectrum (400 MHz, DMSO-d6, δ, ppm, J/Hz): 3.21 (3H, s), 3.30 (3H, s), 3.40 (3H, s), 4.01 (3H, s). ¹³C NMR (400 MHz, DMSO-d6) δ 25.8, 27.4, 28.2, 56.0, 101.5, 142.8, 151.2, 152.0, 154.5. MS(m/z): 225.22 (M+H)⁺. IR: vKBr: 2102, 1717, 1693, 1554, 1519 and 1063 cm⁻¹.

### Example 5: Preparation of 2-Methoxy-1,7,9-trimethyl-7,9-dihydro-1H-purine-6,8-dione (Methylliberine 5)

To the stirred solution of intermediate (4) (600 g, 3.3 mol), obtained in example 3, in acetone (500 mL) was added potassium carbonate (1637 g, 11.86 mol) followed by addition of dimethyl sulfate (1.5 kg, 11.86 mol). The reaction mixture was stirred at 75° C. for 16 h. After 16 h, reaction mixture was cooled to room temperature and filtered. The volatiles in the filtrate were removed under vacuum. The crude was dissolved in MeOH (5 vol) and heated at reflux for 2 h, then cooled at 40° C., solid formed was filtered and washed with hot MeOH (1 vol, 40° C.), to afford Methylliberine (5). Yield: 360 g (49%). ¹HNMR spectrum (400 MHz, DMSO-d6, δ, ppm, J/Hz): 3.21 (3H, s), 3.30 (3H, s), 3.40 (3H, s), 4.01 (3H, s). ¹³C NMR (DMSO-d6, 400 MHz) δ 25.8, 27.4, 28.2, 56.0, 101.5, 142.8, 151.2, 152.0, 154.5. MS (m/z): 225.22 (M+H)⁺. IR: vKBr 2102, 1717, 1693, 1554, 1519 and 1063 cm⁻.

### Example 6: Preparation of 2-Methoxy-1,7,9-trimethyl-7,9-dihydro-1H-purine-6,8-dione (Methylliberine 5)

To the intermediate 4 (500 mg, 2.75 mmol) obtained in example 3, was added DMF-DMA (1.64 mL, 12.38 mmol). The reaction mixture was stirred at 140-145° C. in sealed tube for 8 h. Reaction mixture was cooled to room temperature and added pet-ether (5 mL). The solid formed was filtered and washed with pet ether. Titled compound was dried under vacuum to obtain crude compound, which was crystallized in MeOH to afford Methylliberine (5). Yield 200 mg (33%). ¹HNMR spectrum (400 MHz, DMSO-d6, δ, ppm, J/Hz): 3.21 (3H, s), 3.30 (3H, s), 3.40 (3H, s), 4.01 (3H, s). ¹³C NMR (400 MHz, DMSO-d6) δ 25.8, 27.4, 28.2, 56.0, 101.5, 142.8, 151.2, 152.0, 154.5. MS (m/z): 225.22 (M+H)⁺. IR: vKBr 2102, 1717, 1693, 1554, 1519 and 1063 cm⁻¹.

### Example 7: Preparation of 2-Methoxy-1,7,9-trimethyl-7,9-dihydro-1H-purine-6,8-dione (Methylliberine 5)

To a stirred solution of intermediate 4 (0.5 g, 2.75 mmol), obtained in example 3, in trimethyl phosphate (3.85 g, 27.5 mmol) was added potassium carbonate (1.14 g, 8.25 mmol). The reaction mixture was stirred at 120° C. for 5 h. After 5 h, reaction mixture was cooled to room temperature, added water and extracted with DCM (3×25 mL). The combined organic layers were dried over Na₂SO₄ and concentrated under vacuum. The crude was dissolved in MeOH (5 vol) and heated at reflux for 2 h, then cooled at 40° C. The solid formed was filtered and washed with hot (40° C.) MeOH (1 vol), to afford Methylliberine (5) as white solid. Yield: 220 mg (36%). ¹HNMR spectrum (400 MHz, DMSO-d6, δ, ppm, J/Hz): 3.21 (3H, s), 3.30 (3H, s), 3.40 (3H, s), 4.01 (3H, s). ¹³C NMR (400 MHz, DMSO-d6) δ: 25.8, 27.4, 28.2, 56.0, 101.5, 142.8, 151.2, 152.0, 154.5. MS(m/z): 225.22 (M+H)⁺. IR: vKBr 2102, 1717, 1693, 1554, 1519 and 1063 cm⁻¹.

### Example 8: General Procedure for the Preparation of Methylliberine Polymorphs

(i) Methylliberine (3.0 g) was dissolved in 50 mL of methanol at 60° C., filtered and the filtrate was kept at room temperature for overnight. Crystals formed were filtered and dried under vacuum, to afford Methylliberine polymorph. PXRD: 2⊖ (% relative intensity): 9.31 (0.2); 10.34 (42.5); 10.70 (1.0); 11.21 (100); 12.1 (10.0); 15.09 (0.3); 16.19 (1.2); 18.57 (1.1); 19.86 (0.4); 20.79 (13.3); 21.08 (1.0); 22.54 (1.7); 24.4 (1.2); 25.7 (4.6); 26.92 (16.2); 27.68 (4.9); 28.83 (0.4); 30.10 (0.1); 31.43 (0.6); 34.12 (0.6); 36.69 (0.3); 38.27 (0.5); 39.45 (0.7); 40.87 (0.6); 42.33 (0.3); 43.11 (0.3); 45.57 (0.3).
(ii) Methylliberine (3.0 g) was dissolved in 50 mL of ethanol at 60° C., filtered and the filtrate was kept at room temperature overnight, crystals formed were filtered and dried under vacuum, to afford Methylliberine polymorph. PXRD: 2⊖ (% relative intensity): 9.29 (0.1); 10.32 (21.3); 10.72 (1.0); 11.19 (100); 12.19 (7.6); 14.15 (0.1); 16.18 (1.3); 18.63 (1.3); 19.25 (0.2); 19.84 (0.4); 20.77 (6.8); 21.03 (1.1); 21.61 (0.4); 22.52 (1.6); 23.60 (0.4); 24.41 (1.3); 25.71 (4.3); 26.91 (13.2); 27.70 (4.7); 28.62 (0.6); 28.97 (0.2); 30.66 (0.5); 31.33 (0.4); 31.63 (0.7); 34.09 (0.3); 35.97 (0.2); 36.75 (0.3); 37.63 (0.5); 39.41 (0.6); 40.11 (0.5); 40.83 (0.5); 42.27 (0.3); 43.11 (0.4); 45.48 (0.4); 47.66 (0.2).
(iii) Methylliberine (3.0 g) was dissolved in 60 mL of ethyl acetate at 60° C., filtered and the filtrate was kept at room temperature overnight, crystals formed were filtered and dried under vacuum, to afford Methylliberine polymorph. PXRD: 2⊖ (% relative intensity): 10.23 (22.2); 10.70 (0.9); 11.17 (100); 12.17 (8.1); 15.07 (0.7); 16.17 (0.4); 18.52 (0.3); 20.61 (6.9); 20.82 (5.0); 22.51 (1.7); 24.07 (0.5); 24.37 (0.5); 25.70 (1.3); 26.35 (0.3); 26.90 (4.4); 27.69 (1.5); 31.65 (0.4); 34.04 (0.2); 39.42 (0.4); 40.17 (0.2); 43.08 (0.2); 45.39 (0.1).
(iv) Methylliberine (3.0 g) was dissolved in 30 mL of dichloromethane (DCM) at 60° C., filtered and the filtrate was kept at room temperature overnight, crystals formed were filtered and dried under vacuum, to afford Methylliberine polymorph. PXRD: 2⊖ (% relative intensity): 10.35 (27.7); 10.72 (0.9); 11.19 (100); 12.17 (21.8); 15.09 (1.9); 16.17 (0.8); 16.96 (0.2); 18.58 (0.7); 19.85 (0.2); 20.62 (6.9); 22.53 (1.6); 24.12 (3.4); 24.72 (1.5); 25.70 (2.6); 26.35 (2.9); 26.89 (10.5); 27.70 (2.9); 28.16 (0.4); 28.79 (0.8); 30.64 (0.3); 31.42 (0.6); 31.69 (0.6); 34.11 (0.4); 39.42 (0.5); 40.20 (0.3); 43.09 (0.3).
(v) Methylliberine (3.0 g) was dissolved in 80 mL of Acetone at 60° C., filtered at 60° C., and the filtrate was kept at room temperature overnight, crystals formed were filtered and dried under vacuum, to afford Methylliberine polymorph. PXRD: 2⊖ (% relative intensity): 9.25 (0.6); 10.25 (100); 12.19 (32.5); 15.09 (3.2); 17.00 (0.3); 20.64 (35.6); 22.19 (0.3); 24.13 (2.9); 24.72 (1.9); 26.37 (3.8); 26.65 (1.2); 27.15 (3.7); 28.17 (0.3); 28.84 (0.6); 29.95 (0.2); 31.64 (0.3); 33.11 (0.5); 33.58 (0.5); 35.07 (0.7); 40.04 (0.2); 42.07 (0.3); 43.39 (0.5); 46.67 (0.1); 47.97 (0.1); 48.91 (0.3).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A process for the preparation of methylliberine (5) via the intermediate (4) in an efficient manner comprising;
(a) Reacting 6-amino-2-methoxy-5-nitrosopyrimidin-4-(3H)-one (3) dissolved in formic acid with glyoxilic acid to form the intermediate (3a) which is transformed to intermediate (3b) with subsequent hydroxylation and simultaneous decarboxylation of intermediate (3b) to yield 2-methoxy-1H-purine-6,8-(7H,9H)-dione (4); and
(b) Methylating intermediate (4) in the presence of a base to obtain methylliberine (5).

2. The process as claimed in claim 1, wherein the intermediate 6-amino-2-methoxy-5-nitrosopyrimidin-4-(3H)-one (3) is prepared by the process comprising;
(a) Reacting urea (1) with dimethyl sulfate and sulfuric acid to obtain O-methylisourea hemisulfate (1a), followed by in-situ cyclization with ethyl cyanoacetate to obtain 6-amino-2-methoxy-pyrimidin-4-(3H)-one (2); and
(b) Nitrosating 6-amino-2-methoxy-pyrimidin-4-(3H)-one (2) to obtain 6-amino-2-methoxy-5-nitrosopyrimidin-4-(3H)-one (3).

3. The process for the preparation of methylliberine (5) in an efficient manner as claimed in claim 1 comprising;
(a) Reacting urea (1) with dimethyl sulfate and sulfuric acid to obtain O-methylisoureahemisulfate (1a), followed by in-situ cyclization with ethyl cyanoacetate to obtain 6-Amino-2-methoxy-pyrimidin-4-(3H)-one 2;
(b) Nitrosating 6-amino-2-methoxy-pyrimidin-4(3H)-one 2 to obtain 6-amino-2-methoxy-5-nitrosopyrimidin-4-(3H)-one 3;
(c) Reacting 6-amino-2-methoxy-5-nitrosopyrimidin-4-(3H)-one (3) dissolved in formic acid with glyoxilic acid to form the intermediate (3a) which is transformed to intermediate (3b) with subsequent hydroxylation and simultaneous decarboxylation of intermediate (3b) to yield 2-methoxy-1H-purine-6,8(7H,9H)-dione (4); and
(d) Methylating intermediate (4) in presence of base to obtain methyl liberine (5).

4. The process as claimed in claim 1, wherein the methylating agent is selected from dimethyl sulfate, dimethyl carbonate, N,N-dimethylformamide-dimethylacetal (DMF-DMA) or trimethyl phosphate alone or mixtures thereof; the base is selected from alkali or alkaline metal hydroxides, carbonates, bicarbonates; alkali or alkaline metal methoxides; diethylamine, triethylamine, diisopropylethylamine or pyridine or 1,8-diazabicyclo [5.4.0] undec-7-ene (DBU) or 1,5-diazabicyclo[4.3.0]non-5-one.

5. The process as claimed in claim 1, wherein the solvent is selected from water, organic solvents such as DMF, DMA, ketones, ethers, esters, lower aliphatic or aromatic hydrocarbons, lower alcohols alone or mixtures thereof.

6. (canceled)

7. The compound 6-amino-2-methoxy-5-nitrosopyrimidin-4-(3H)-one of formula (3),

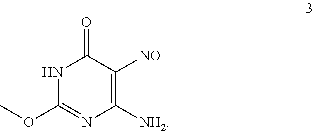

8. The compound 2-methoxy-1H-purine-6,8-(7H,9H)-dione of the formula (4),

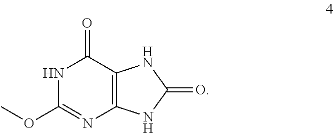

9. A process for the preparation of methylliberine (5) polymorphs comprising crystallization of methylliberine (5) from the solvent selected from water, lower alcohols such as ethanol, methanol, butanol; halogenated hydrocarbons such as methylene dichloride, ethylene dichloride; ketones such as acetone; ethers such as diethtylether, THF; hydrocarbons such as hexane, heptanes, toluene, xylene; and such like alone or mixtures thereof.

10. The process as claimed in claim 9, wherein the polymorphs are characterized by the PXRD 2⊖ (% relative intensity) peaks at;
(i) 9.31 (0.2); 10.34 (42.5); 10.70 (1.0); 11.21 (100); 12.1 (10.0); 15.09 (0.3); 16.19 (1.2); 18.57 (1.1); 19.86 (0.4); 20.79 (13.3); 21.08 (1.0); 22.54 (1.7); 24.4 (1.2); 25.7 (4.6); 26.92 (16.2); 27.68 (4.9); 28.83 (0.4); 30.10 (0.1); 31.43 (0.6); 34.12 (0.6); 36.69 (0.3); 38.27 (0.5); 39.45 (0.7); 40.87 (0.6); 42.33 (0.3); 43.11 (0.3); 45.57 (0.3);
(ii) 9.29 (0.1); 10.32 (21.3); 10.72 (1.0); 11.19 (100); 12.19 (7.6); 14.15 (0.1); 16.18 (1.3); 18.63 (1.3); 19.25 (0.2); 19.84 (0.4); 20.77 (6.8); 21.03 (1.1); 21.61 (0.4); 22.52 (1.6); 23.60 (0.4); 24.41 (1.3); 25.71 (4.3); 26.91 (13.2); 27.70 (4.7); 28.62 (0.6); 28.97 (0.2); 30.66 (0.5); 31.33 (0.4); 31.63 (0.7); 34.09 (0.3); 35.97 (0.2); 36.75 (0.3); 37.63 (0.5); 39.41 (0.6); 40.11 (0.5); 40.83 (0.5); 42.27 (0.3); 43.11 (0.4); 45.48 (0.4); 47.66 (0.2);
(iii) 10.23 (22.2); 10.70 (0.9); 11.17 (100); 12.17 (8.1); 15.07 (0.7); 16.17 (0.4); 18.52 (0.3); 20.61 (6.9); 20.82 (5.0); 22.51 (1.7); 24.07 (0.5); 24.37 (0.5); 25.70 (1.3); 26.35 (0.3); 26.90 (4.4); 27.69 (1.5); 31.65 (0.4); 34.04 (0.2); 39.42 (0.4); 40.17 (0.2); 43.08 (0.2); 45.39 (0.1);
(iv) 10.35 (27.7); 10.72 (0.9); 11.19 (100); 12.17 (21.8); 15.09 (1.9); 16.17 (0.8); 16.96 (0.2); 18.58 (0.7); 19.85 (0.2); 20.62 (6.9); 22.53 (1.6); 24.12 (3.4); 24.72 (1.5); 25.70 (2.6); 26.35 (2.9); 26.89 (10.5); 27.70 (2.9); 28.16 (0.4); 28.79 (0.8); 30.64 (0.3); 31.42 (0.6); 31.69 (0.6); 34.11 (0.4); 39.42 (0.5); 40.20 (0.3); 43.09 (0.3);
(v) 9.25 (0.6); 10.25 (100); 12.19 (32.5); 15.09 (3.2); 17.00 (0.3); 20.64 (35.6); 22.19 (0.3); 24.13 (2.9); 24.72 (1.9); 26.37 (3.8); 26.65 (1.2); 27.15 (3.7); 28.17 (0.3); 28.84 (0.6); 29.95 (0.2); 31.64 (0.3); 33.11 (0.5); 33.58 (0.5); 35.07 (0.7); 40.04 (0.2); 42.07 (0.3); 43.39 (0.5); 46.67 (0.1); 47.97 (0.1); 48.91 (0.3).
